(19) United States
(12) Patent Application Publication
(10) Pub. No.: US 2021/0094034 A1
(43) Pub. Date: Apr. 1, 2021

(54) Modular Microfluidic Assay System

(71) Applicant: National University of Singapore, Singapore (SG)

(72) Inventor: Luke P. LEE, Singapore (SG)

(21) Appl. No.: 16/620,442

(22) PCT Filed: Jun. 7, 2018
(86) PCT No.: PCT/SG2018/050287

§ 371 Date: Dec. 6, 2019

Related U.S. Application Data
(60) Provisional application No. 62/516,724, filed on Jun. 8, 2017.

(51) Int. Cl. B01L 3/00; C12M 3/06; C12M 3/00

(52) U.S. Cl. CPC B01L 3/502715; C12M 23/16; C12M 23/42; C12M 23/44; B01L 2300/0829; B01L 2200/028; B01L 2200/0647; B01L 2300/0645; B01L 2200/027

(57) ABSTRACT

A modular microfluidic system can include an individual microfluidic module that can be coupled to and decoupled from a socket. The socket can be coupled to a fluid source and electrical circuitry. A fluid interface between the socket and the module can allow fluid flow between the socket and the module. The module can include any number of wells or other microfluidic features. In some cases, the wells of the module can fit within corresponding recesses of the socket. In some cases, an optional electrical interface may provide electrical connection between the socket and the module. In some cases, the electrical circuitry is coupled to electrodes within the socket, such as electrodes positioned adjacent recesses. The module is designed for easy removal from and placement on the socket, allowing assays to be rapidly deployed, moved, and removed.

### TECHNICAL FIELD

The present disclosure relates to fluidic diagnostic systems generally and more specifically to microfluidic systems.

### BRIEF DESCRIPTION OF THE DRAWINGS

The specification makes reference to the following appended figures, in which use of like reference numerals in different figures is intended to illustrate like or analogous components.

FIG. 1 is a schematic diagram of a modular microfluidic assay system according to certain aspects of the present disclosure.

FIG. 2 is a combination flowchart and schematic diagram depicting a process for using a modular microfluidic assay system according to certain aspects of the present disclosure.

FIG. 3 is a partially see-through, axonometric view depicting a modular microfluidic assay system according to certain aspects of the present disclosure.

FIG. 4 is a close-up, partially see-through, axonometric view depicting the modular microfluidic assay system of FIG. 3 according to certain aspects of the present disclosure.

FIG. 5 is a close-up, partially see-through, partial-cut-away axonometric view depicting the modular microfluidic assay system of FIG. 3 according to certain aspects of the present disclosure.

FIG. 6 is an isometric view depicting a modular microfluidic assay system according to certain aspects of the present disclosure.

FIG. 7 is a side view depicting a modular microfluidic assay system according to certain aspects of the present disclosure.

FIG. 8 is a cutaway side view depicting fluid interfaces of a microfluidic assay system according to certain aspects of the present disclosure.

FIG. 9 is an exploded view of the cutaway side view of the microfluidic assay system of FIG. 8 according to certain aspects of the present disclosure.

FIG. 10 is a cutaway side view depicting chambers of a module of a microfluidic assay system according to certain aspects of the present disclosure.

FIG. 11 is an exploded view of the cutaway side view of the microfluidic assay system of FIG. 10 according to certain aspects of the present disclosure.

FIG. 12 is a cutaway side view of a receptacle of a socket with subsurface electrodes according to certain aspects of the present disclosure.

FIG. 13 is a cutaway side view of a receptacle of a socket with trans-surface electrodes according to certain aspects of the present disclosure.

FIG. 14 is a cutaway side view of a receptacle of a socket with subsurface electrode arrays and a view port according to certain aspects of the present disclosure.

FIG. 15 is a cutaway side view of a receptacle of a socket with trans-surface electrode arrays and a view port according to certain aspects of the present disclosure.

### DETAILED DESCRIPTION

Certain aspects and features of the present disclosure relate to a modular microfluidic system. The system can include an individual microfluidic module that can be coupled to and decoupled from a socket. The socket can be coupled to a fluid source and electrical circuitry. A fluid interface between the socket and the module can allow fluid flow between the socket and the module. The module can include any number of chambers or other microfluidic features. In some cases, the chambers of the module can fit within corresponding recesses of the socket. In some cases, an optional electrical interface may provide electrical connection between the socket and the module. In some cases, the electrical circuitry is coupled to electrodes within the socket, such as electrodes positioned adjacent recesses. The module is designed for easy removal from and placement on the socket, allowing assays to be rapidly deployed, moved, and removed.

The socket can include a main body containing microfluidic passageways and electrical lines. Fluids can be exchanged (e.g., either entering or exiting the socket) with a fluid source via fluid ports (e.g., inlets and/or outlets). Fluid ports can be located at any suitable location, such as at the periphery of the socket. One or more fluid interface elements of the socket can interact with one or more fluid interface elements of the module to exchange fluid between fluid passageways within the module and the fluid passageways of the socket. The fluid interfaces can take any suitable shape or form. In an example, the socket can have a fluid interface element that is a rigid passageway extending upwards from a top surface of the socket. The module can have a corresponding fluid interface element that is a recessed passageway into which the rigid passageway can fit to fluidly couple the socket with the module. In some cases, washers or gaskets can be used to ensure a fluid-tight seal. In some cases, the module can be made of an elastomeric material suitable for creating a fluid-tight seal without additional gaskets. In another example, the module may have a rigid passageway extending downwards from a bottom surface of the module that interacts with a corresponding recess of the socket to form a fluid interface. Other fluid interfaces can be used.

The module can include one or more chambers or other microfluidic features. The socket can include recesses that correspond with any chambers or other microfluidic features of the module. In some cases, a module can have an array of chambers and the socket can have an array of recesses, with each chamber designed to abut and/or fit at least partially within respective recesses. Electrodes can be positioned adjacent each recesses to allow electrical signals to be provided to or sensed from the contents of the corresponding chamber. For example, electrodes can provide stimuli to organoids within in the chambers. In another example, electrodes can be used to take electroencephalograms of cerebral organoids within the chambers.

In some cases, the socket can be made from materials suitable for electrical conduction and the electrodes can be located under the surface of the recess. In some cases, the electrodes may be exposed through the surface of the recess. The electrodes can sense electrical activity of the contents of the chambers and/or can provide electrical stimulus to the contents of the chambers. Electrodes can be individually addressable, allowing each chamber to be monitored and/or controlled individually. Electrodes and corresponding electrical conductors (e.g., wires or electrical paths) within the socket can be routed to avoid areas directly below a recess, such as to avoid inhibiting visual inspection of the chamber (e.g., through bright-field microscopy). For example, electrodes and conductors can be routed in location between receptacles (e.g., between chambers). In some cases, electrodes and/or corresponding electrical conductors can be made of a transparent or sufficiently translucent material, such as indium tin oxide (ITO).

In some cases, one or more optional electrical interfaces can provide electrical communication between the socket and the module, such as to power electrical devices within the module (e.g., pumps, heating elements, lights) or otherwise interact with microfluidic elements of the module (e.g., sensing electrical activity or providing electrical stimulus).

In some cases, the socket can include additional electrical devices. For example, the socket can include lights to illuminate the chambers, heaters to provide temperature control to the chambers, or sensors (e.g., thermocouples) to sense parameters of the chambers.

In some cases, a module can include microfluidic elements other than chambers, such as elongated diffusion channels or other desirable elements. The socket can include corresponding features for these elements, such as recesses into which elongated diffusion channels can rest. These recesses can have electrodes similarly to recesses associated with chambers. In some cases, the socket can contain more recesses than corresponding features of a module.

For example, a single socket can include a first set of recesses for an array of chambers and second set of recesses for other microfluidic elements, such as diffusion channels. Thus, modules having chambers and modules having diffusion channels can be used with the same socket. In some cases, the first set of recesses can partially overlap with the second set of recesses.

Unlike microfluidic devices with integrated electrodes (e.g., electrodes built into or around the microfluidic wells of a microfluidic array), certain aspects and features of the present disclosure provide for a modular microfluidic system wherein electronics (e.g., electrodes) are located in a reusable socket and microfluidics are located in a fluidic module, such as a disposable fluidic module.

The modular nature of the microfluidic system can allow modules to be rapidly deployed and easily switched out as needed. Modules can be identical, similar, or dissimilar. For example, different modules can be provided that have different microfluidic elements arranged in different fashions, however each module can have the same fluid interface, allowing the module to interact with the socket. In some cases, different modules can all have a similar external form factor and placement of fluid interface.

In some cases, a particular module design (e.g., arrangement of chambers or other microfluidic elements) can be associated with configuration data for configuring how fluid is pumped through the socket and/or how electrical signals are received and/or sent within the socket. For example, a particular module design may require that certain fluid ports of the socket are set up as inlets (e.g., for flowing fluid into socket and thereafter into the module) and other fluid ports are set up as outlets (e.g., for flowing fluid from the module and out through the socket). Configuration data can include fluidic configuration data which can define parameters related to the fluidic operations of the socket and/or module. Fluidic configuration data can define which fluid ports of the socket are to function as inlets and outlets, which fluid ports may function as control channels, or any other characteristics of the fluid ports (e.g., minimum and maximum pressures, flow speeds, text labels, or other data). Configuration data can include electrical configuration data which can define parameters related to the electrical operations of the socket and/or module. Electrical configuration data can define which electrical ports of the socket function as inputs or outputs, sensitivity settings for outputs, control settings for inputs, or any other characteristics of the electrical ports (e.g., electrical properties, text labels, or other data). Configuration data can be associated with particular module designs. A processor or other controller associated with the microfluidic system (e.g., a processor coupled to a microfluidic pump to control fluid flow through the socket or a processor coupled to electrical circuitry used to sense electrical signals in the socket or transmit electrical stimuli to chambers of the modules) can use the configuration data to load presets associated with a particular microfluidic module, thereby making setup easier and faster.

The module can be made of any suitable material, such as polydimethylsiloxane (PDMS) or a suitable plastic (e.g., polycarbonate). The module can be transparent to facilitate visual inspection of the microfluidic device and/or the contents of any chambers. As used herein, the term transparent can mean fully transparent and at least sufficiently translucent to perform the desired assays. The socket can be made of similar or different materials than the module. In some cases, the socket can be made from PDMS or a suitable plastic. The socket can be transparent to facilitate visual inspection of the module.

In some cases, the microfluidic module and socket are designed for easy manual coupling and decoupling without the need for tools or additional parts. In some cases, the fluid interface(s) can provide sufficient mechanical coupling to help retain the module on the socket and inhibit inadvertent decoupling of the module from the socket. In such cases, the fluid interface(s) can provide both fluidic coupling and mechanical coupling of the module to the socket. In some cases, other mechanical couplings can be used to secure the module to the socket, such as external frames used to compress the module against the socket.

The modular microfluidic assay system can be made in any suitable scale. In one example, a socket and corresponding modules can have locations for 100 chambers. In another example, the socket and corresponding modules can have locations for 1000 chambers or more.

The modular nature of the microfluidic system can allow a module to be removed from a first socket during an experiment and coupled to a different socket to continue the experiment.

The modular microfluidic assay system can be especially useful for research associated with organoids (e.g., minibrains or cerebral organoids), dynamic cell cultures, and other microfluidic assays. The modular nature allows new assays to be rapidly deployed without needing to outfit existing microfluidic chips with electrodes. Further, the re-use of electrodes in the socket can allow the microfluidic module to be mass-produced at lower cost. Additionally, the constant position of the electrodes with respect to the socket recesses, and therefore the chambers of the modules, can provide improved consistency between subsequent assays.

These illustrative examples are given to introduce the reader to the general subject matter discussed here and are not intended to limit the scope of the disclosed concepts. The following sections describe various additional features and examples with reference to the drawings in which like numerals indicate like elements, and directional descriptions are used to describe the illustrative embodiments but, like the illustrative embodiments, should not be used to limit the present disclosure. The elements included in the illustrations herein may not be drawn to scale.

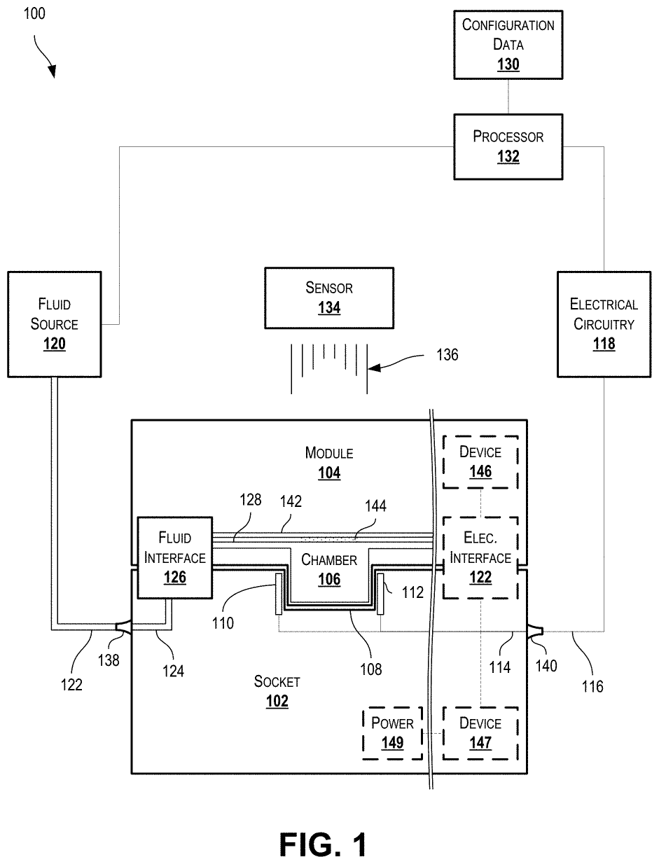

FIG. 1 is a schematic diagram of a modular microfluidic assay system 100 according to certain aspects of the present disclosure. The system 100 can include a socket 102 and a module 104. The module 104 can include a chamber 106 (e.g., well) that fits adjacent a receptacle 108 of the socket 102. The receptacle 108 can be a recess into which the chamber 106 fits, although the receptacle 108 and chamber 106 can take other shapes, such as flat surfaces or a receptacle of a module can extend into a recess of a chamber. As depicted in FIG. 1, the receptacle 108 is a recess in a top surface of the socket 102 into which the chamber 106 of the module 104 rests. The receptacle 108 can have a shape that corresponds to the shape of the chamber 106 such that surfaces of the chamber 106 rest flush against surfaces of the receptacle 108. As used herein, a chamber 106 can refer to a well, a channel, or other microfluidic element suitable for microfluidic assay. A gap is depicted between the walls of the chamber 106 and the receptacle 108 for clarity, however the chamber 106 may rest flush against the receptacle 108.

The socket 102 can be coupled to one or more fluid sources 120 through one or more external fluidic passageways 122. A fluid source 120 can include a supply of fluid and optionally a source of pressure differential (e.g., pressure source or vacuum source) to facilitate moving fluid through the socket 102 and module 104. External fluidic passageways 122 can be piping, tubing, or any suitable passage for transmitting fluids. The external fluidic passageways 122 can couple to socket 102 at one or more fluid ports 138. Fluid can be transferred between the socket 102 and the module 104 through a fluid interface 126. Each fluidic port 138 of the socket 102 can be coupled to the fluid interface 126 via internal fluidic passageways 124 of the socket 102. The fluid interface 126 of the module 104 can be coupled to one or more chambers 106 through internal fluidic channels 128 of the module 104. The fluid passageways 122, 124, fluid channels 128, ports 138, chambers 106, and fluid interfaces 126 can define fluid paths into the socket 102, through the socket 102, into the module 104, through the module 104, out of the module 104, back into the socket 102, and out of the socket 102.

Fluid paths can be circular (e.g., supplying fluid flow) or one-way (e.g., supplying pressure). A circular fluid path can flow a volume of fluid through the socket 102 and module 104 and generally require at least one port 138 to act as an input (e.g., supply) and at least one port 138 to act as an output (e.g., return). Optionally, multiple circular fluid paths can share a common input and/or output. A one-way fluid path can supply pressure through passageways 122, 124 and fluid channels 128, such as to control a pressure-sensitive feature or element of the module 104.

The system 100 depicted in FIG. 1 is an example of a system useful for developing and/or testing organoids. The module 104 includes one or more chambers 106 coupled to one or more fluidic channels 128 that are fluidly coupled to the fluid source 120 through one or more fluid interfaces 126 with the socket 102. The module 104 can also include one or more perfusion channels 142 located adjacent the chambers 106 to provide perfusion to the contents of the chambers 106 through perfusion regions 144. The perfusion channels 142 can be fluidly coupled to the fluid source 120 through one or more fluid interfaces 126 with the socket 102. Controlling fluid supplied to the socket through ports 138 can control fluid passing through the fluidic channels 128 and perfusion channels 142 of the module 104.

The socket 102 can further include one or more electrodes 110, 112 located at or adjacent the receptacle 108. The electrodes 110, 112 can be positioned to be at least partially electrically coupled with the chamber 106 when the module 104 is in place on the socket 102, such as to sense electrical activity within chamber 106 or to provide electrical signals to the contents of the chamber 106. In some cases, electrodes 110, 112 can be uniquely addressable for each receptacle 108. In some cases, electrode 110 is a common electrode that is common for each receptacle 108, while electrode 112 can be uniquely addressable for each receptacle 108. In some cases, more than one electrode is provided at or adjacent a receptacle 108. Electrodes 110, 112 can be coupled to one or more electrical ports 140 of the socket through internal conductors 114. Electrical circuitry 118 can be coupled to the ports 140 of the socket 102 using external conductors 116 (e.g., cables or wires) to access the electrodes 110, 112. Electrical circuitry 118 can be any suitable circuitry, including sensing equipment (e.g., electroencephalography sensing equipment), pulse generators, or other electrical equipment.

In some cases, an electrical interface 122 can optionally be included to electrically couple socket 102 to module 104. In such cases, an electrical device 146 within the module can be controlled, powered, or accessed through ports 140 of the socket 102 via electrical interface 122. For example, an electrical device 146 that is a light (e.g., light emitting diode) can be powered and controlled by the electrical circuitry 118 through port 140 and electrical interface 122. In another example, an electrical device 146 that is an additional electrode can be accessed by the electrical circuitry 118 through port 140 and electrical interface 122. Such an example can provide for improved three-dimensional differentiation of electrical signals when used in conjunction with electrodes 110, 112 and when the additional electrode (e.g., device 146) is located within the module 104 and adjacent chamber 106. Any other suitable electrical device 146 can be used within module 104.

Module 104 and socket 102 can be made of any suitable material, such as PDMS. Module 104 can be transparent such that the chamber 106 can be visually inspected. In some cases, socket 102 can be transparent to facilitate visual inspection of chamber 106, such as by allowing light to be transmitted through socket 102 and through chamber 106. In some cases, a sensor 134, such as an optical sensor (e.g., digital microscope) can detect radiation 136 (e.g., light) emitted from, transmitted through, or reflected by the contents of chamber 106.

One or more processors 132 can control the fluid source 120 and/or the electrical circuitry 118. For example, a processor 132 can control the flow rates of any fluids flowing through the external fluid passageways 122, either two or from the socket 102. In another example, processor 132 can control the sensing of electrical activity in the chamber 106 through electrodes 110, 112. In another example, processor 132 can control the transmission of electrical stimuli to the chamber 106 through electrodes 110, 112.

Processor 132 can be coupled to a data storage for storing configuration data 130. Configuration data 130 can include any parameters or other data associated with the processor's 132 control over the system 100, such as fluid flow rates, identification labels and settings for fluid ports 138, electrical functions, identification labels and settings for electrical ports 140, or any other such data. In some cases, configuration data 130 can be associated with a particular experiment being conducted. In some cases, configuration data 130 can be associated with a particular module 104, such that different styles of modules may be associated with different sets of configuration data. In some cases, a sensor associated with the processor 132 can automatically determine what configuration data 130 to load or suggest for use by identifying the type of module 104 coupled to the socket 102.

In some cases, an optional device 147 can be located in the socket 102 for performing various functions powered by or controllable through electrical signals passing through ports 140. An optional power source 149 (e.g., a battery or ultra-capacitor) can be coupled to device 147 to provide power to the device 147 as necessary. In some cases, the optional device 147 can include one or more sensors for sensing information relevant to the socket, the module, or one or more chambers 106. For example, a sensor can detect and/or monitor temperature, pressure, vibrations, movement, light intensity, magnetic fields, or other data. In some cases, optional device 147 is capable of processing data and transmitting data wirelessly, without requiring the use of electrical ports 140. For example, optional device 147 can include electrical circuitry, a processor, and a wireless transmitter that allows the system 100 to operate without external electrical connections. In such cases, fluidic passageways 122 can be coupled to the fluid ports 138 and no electrical conductors 116 may be coupled to the electrical ports 140, with the electrodes 110, 112 and optional device 146 being powered and operated from the optional device 147 and optional power source 149. Thus, the system 100 can be located in a conventional incubator with automated fluidic controls and still provide electrical data collection or electrical interactions wirelessly. For example, the device 147 can send and/or receive wireless signals to and/or from a computer.

In some cases, device 147 can include one or more integrated amplifiers, multiplexers, analog-to-digital converters, and the like for interacting with the various electrical components of the socket 102. In some cases, device 147 can perform the functions of the electrical circuitry 118 and can be coupled directly to processor 132. In some cases, device 147 can perform the functions of both the electrical circuitry 118 and the processor 132 (e.g., at least with respect to the electrical and non-fluidic aspects of the socket 102).

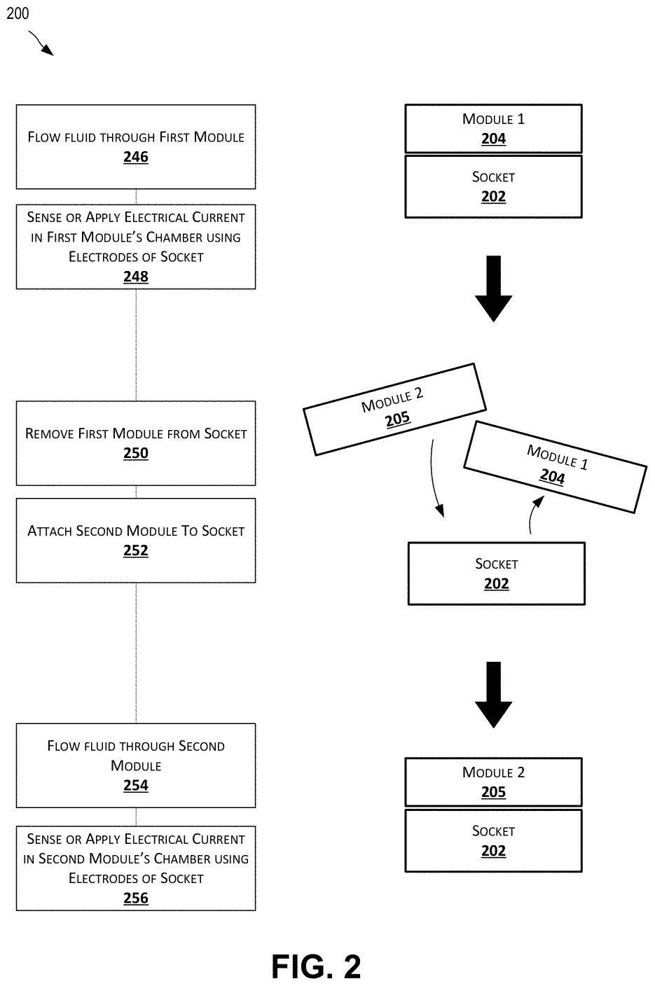

FIG. 2 is a combination flowchart and schematic diagram depicting a process 200 for using a modular microfluidic assay system according to certain aspects of the present disclosure. At block 246, fluid is flowed through the first module 204 from the socket 202. At block 248, electrical current is sensed or applied in the chamber of the first module 204 using electrodes of the socket 202. At block 250, the first module 204 is removed from the socket 202. At block 252, the second module 205 is coupled to the socket 202. At block 246, fluid is flowed through the second module 205 from the socket 202. At block 248, electrical current is sensed or applied in the chamber of the second module 205 using electrodes of the socket 202.

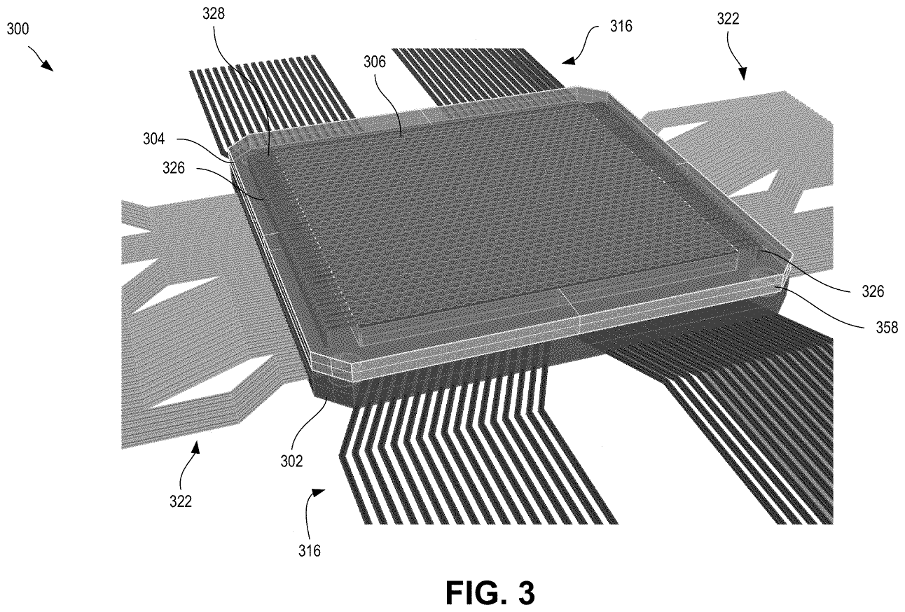

FIG. 3 is a partially see-through, axonometric view depicting a modular microfluidic assay system 300 according to certain aspects of the present disclosure. The system 300 can include a socket 302 and a module 304. Socket 302 and module 304 can be similar to socket 102 and module 104 of FIG. 1. The socket 302 can include a number of external fluid passageways 322 entering the socket 302 at a number of fluid ports. The fluid ports can be located at or near the periphery of the socket 302. In some cases, the fluid ports are located on opposite sides of the socket 302. The socket 302 can include a number of external conductors 316 coupled to the socket 302 at electrical ports. The electrical ports can be located at or near the periphery of the socket 302. In some cases, the electrical ports are located on opposite sides of the socket 302. In some cases, the electrical ports are located on different sides of the socket 302 from fluid ports.

Module 104 can rest on an upper surface of socket 302. One or more fluidic interfaces 326 can couple the fluid channels 328 of the module 304 to the socket 302, and thus the external fluid passageways 322. The module 304 can include a number of chambers 306. In some cases, the fluidic interfaces 326 can mechanically support the module 304 and secure the module 304 from inadvertent disconnection with the socket 302. In some cases, the socket 302 and module 304 can include mechanical features 358. The mechanical features 358 can interlock to facilitate alignment of the module 304 over the socket 302. In some cases, the mechanical features 358 can interlock to promote securing the module 304 to the socket 302 and inhibit inadvertent disconnection of the module 304 from the socket 302.

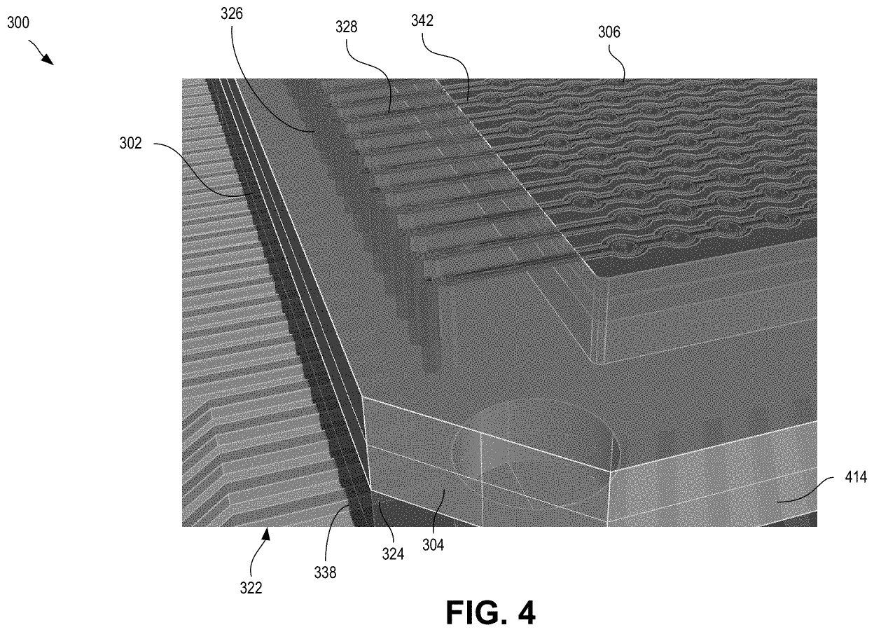

FIG. 4 is a close-up, partially see-through, axonometric view depicting the modular microfluidic assay system 300 of FIG. 3 according to certain aspects of the present disclosure. Multiple external fluid passageways 322 are seen entering the socket 302 at ports 338. Internal fluid passageways 324 within the socket 302 fluidly couple the external fluid passageways 322 to the module's 304 fluid channels 328, including perfusion channels 342, via fluid interfaces 326.

Multiple chambers 306 are depicted in the module 304. Electrodes (not shown) can be coupled to internal conductors 414 and can be positioned in the socket 302 at locations adjacent the chambers 306.

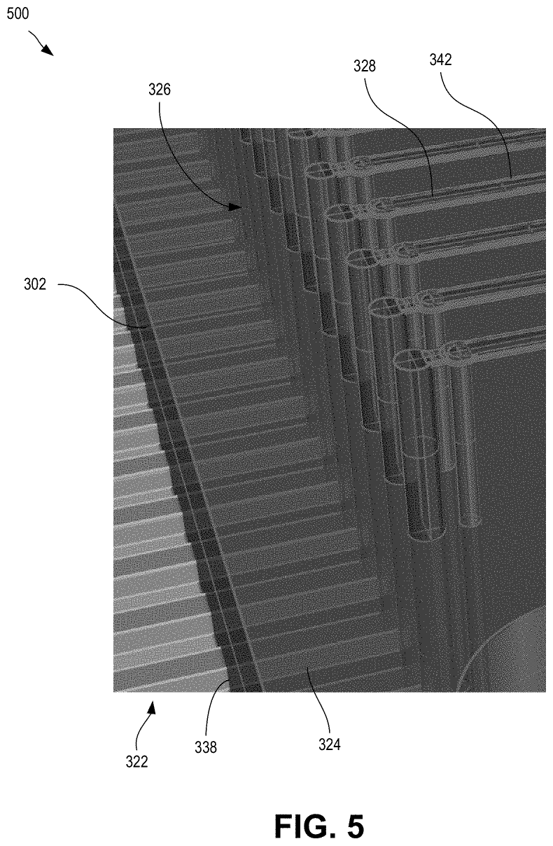

FIG. 5 is a close-up, partially see-through, partial-cut-away axonometric view depicting the modular microfluidic assay system 300 of FIG. 3 according to certain aspects of the present disclosure. As depicted in FIG. 5, the main body of the module is cut away, showing only the fluid channels 328, including the perfusion channels 342, of the module. The fluid interfaces 326 between the module and the socket 302 can be seen coupling the fluid channels 328 of the module with the internal fluid passageways 324 of the socket 302, the fluid ports 338 of the socket 302, and the external fluid passageways 322.

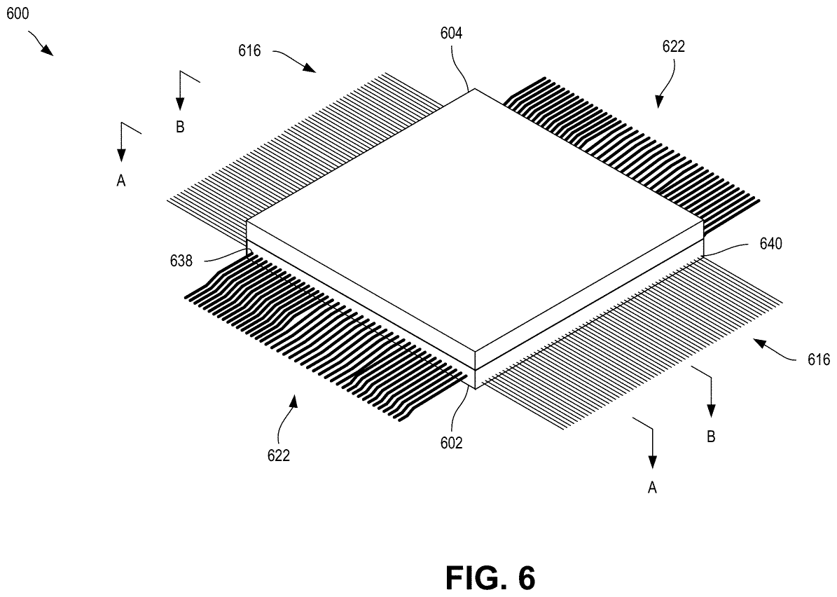

FIG. 6 is an isometric view depicting a modular microfluidic assay system 600 according to certain aspects of the present disclosure. The system 600 can include a socket 602 and a module 604. Internal structures of the socket 602 and module 604 are not shown for illustrative purposes. Multiple external fluid passageways 622 are depicted coupled to the socket 602 at fluid ports 638. Multiple external conductors 616 are depicted coupled to the socket 602 at electrical ports 640. Line A:A defines a plane through the fluid interface between the socket 602 and the module 604. Line B:B defines a plane through a set of chambers of the module 604.

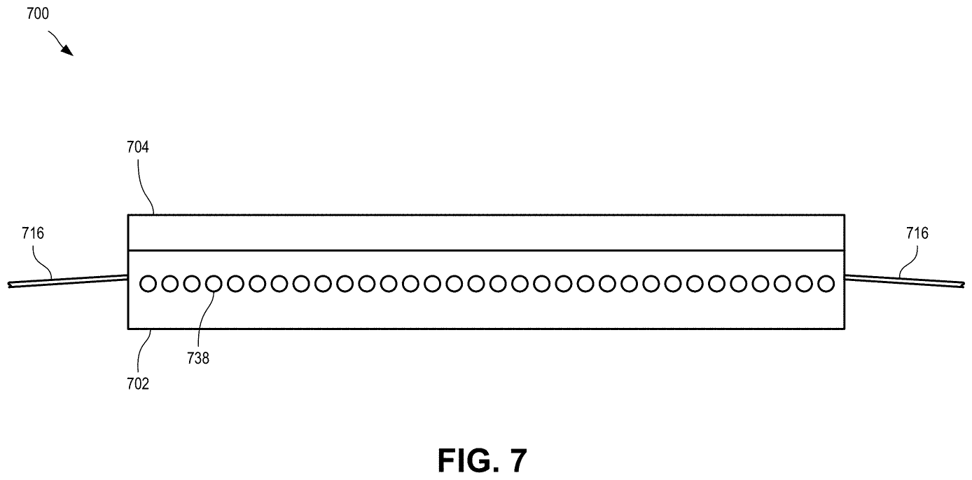

FIG. 7 is a side view depicting a modular microfluidic assay system 700 according to certain aspects of the present disclosure. System 700 can be similar to system 600 of FIG. 6. The system 700 includes a module 704 coupled to a socket 702. Multiple external conductors 716 are coupled to the socket 702 at electrical ports. Multiple external fluid passageways (not shown) can couple to the socket 702 at fluid ports 738.

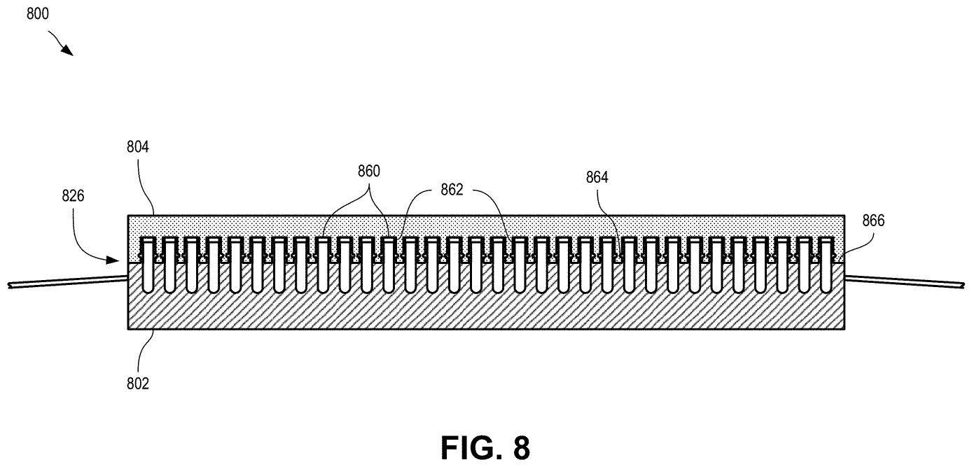

FIG. 8 is a cutaway side view depicting fluid interfaces 826 of a microfluidic assay system 800 according to certain aspects of the present disclosure. System 800 can be similar to system 600 of FIG. 6. The cutaway view of FIG. 8 can be taken along a line similar to line A:A of FIG. 6. The system 800 can include a module 804 coupled to a socket 802.

Fluid interfaces 826 can couple together internal fluid passageways of the socket 802 with fluid channels of the module 804. The fluid interfaces 826 can take various forms. As depicted in FIG. 8, the socket 802 can include a number of rigid passageways 862 extending upwards from an upper surface 866 of the socket 802. Each rigid passageway 862 can be in fluid communication with a fluid port of the socket 802. The rigid passageways 862 of socket 802 can be inserted within recessed passageways 860 of the module 804. Once the rigid passageways 862 are inside the recessed passageways 860, fluid can flow therebetween. In some cases, gaskets or other materials can be added to ensure a fluid-tight seal between the rigid passageways 862 and the recessed passageways 860. In some cases, the flexibility of the module 804 can facilitate creating a fluid-tight seal. In some cases, a protuberance 864 around the exterior of the rigid passageways 862 can facilitate forming a fluid-tight seal with the recessed passageways 862. Rigid passageways 862 can be formed of any suitable material. In some cases, rigid passageways 862 can be formed of a firm plastic.

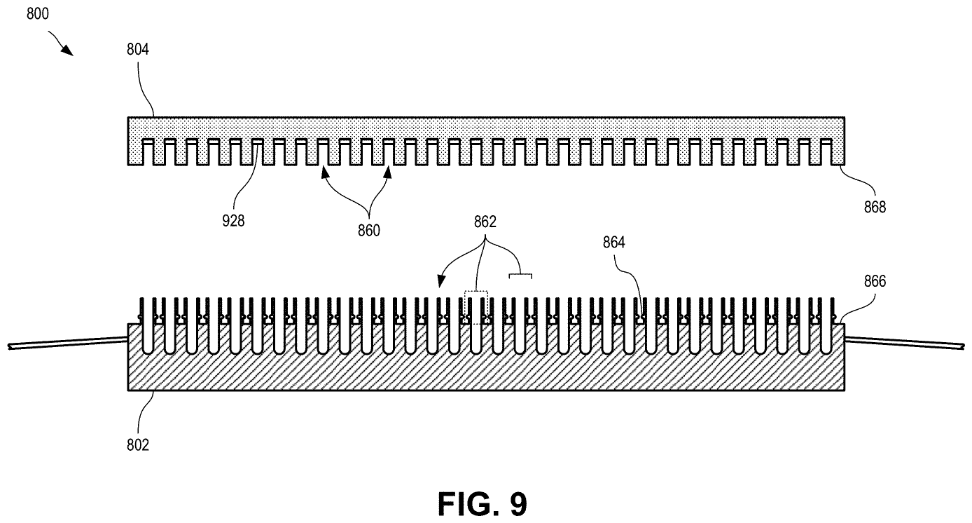

FIG. 9 is an exploded view of the cutaway side view of the microfluidic assay system 800 of FIG. 8 according to certain aspects of the present disclosure. Socket 802 can include a number of rigid passageways 862 extending upwards from an upper surface 866 of the socket 802. Each rigid passageway 862 can be in fluid communication with a fluid port of the socket 802. The rigid passageways 862 of socket 802 can be inserted within recessed passageways 860 of the module 804. Once the rigid passageways 862 are inside the recessed passageways 860, fluid can flow therebetween. In some cases, gaskets or other materials can be added to ensure a fluid-tight seal between the rigid passageways 862 and the recessed passageways 860. In some cases, the flexibility of the module 804 can facilitate creating a fluid-tight seal. In some cases, a protuberance 864 around the exterior of the rigid passageways 862 can facilitate forming a fluid-tight seal with the recessed passageways 862.

The recessed passageways 860 can be recessed into a lower surface 968 of module 804. Each recessed passageway 860 can be coupled to a fluid channel 928 of the module 804.

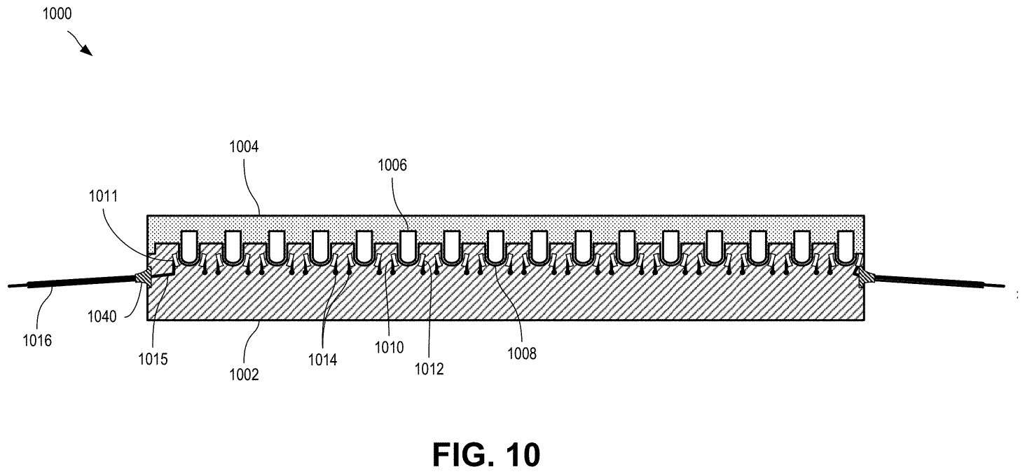

FIG. 10 is a cutaway side view depicting chambers 1006 of a module 1004 of a microfluidic assay system 1000 according to certain aspects of the present disclosure. System 1000 can be similar to system 600 of FIG. 6. The cutaway view of FIG. 10 can be taken along a line similar to line B:B of FIG. 6. The system 1000 can include a module 1004 coupled to a socket 1002.

Socket 1002 can include an array of receptacles 1008 into which the chambers 1006 of module 1004 can fit. Each receptacle 1008 can include electrodes 1010, 1012 that can be coupled to various external conductors via internal conductors 1014. For example, electrode 1011 can be coupled to external conductor 1016 via internal conductor 1015 and electrical plug 1040.

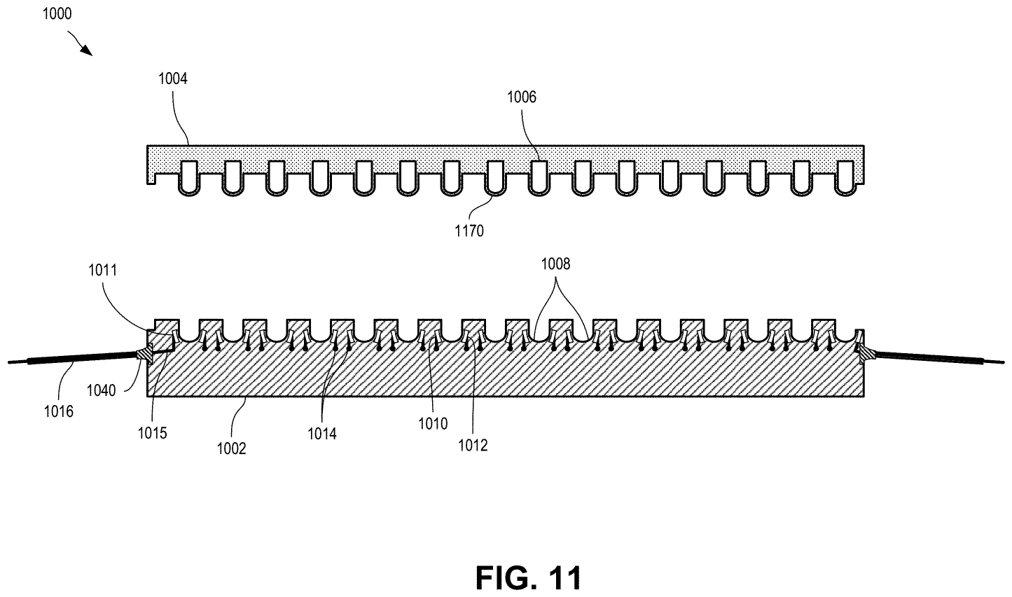

FIG. 11 is an exploded view of the cutaway side view of the microfluidic assay system 1000 of FIG. 10 according to certain aspects of the present disclosure. Socket 1002 can include an array of receptacles 1008 into which the chambers 1006 of module 1004 can fit. Each receptacle 1008 can include electrodes 1010, 1012 that can be coupled to various external conductors via internal conductors 1014. For example, electrode 1011 can be coupled to external conductor 1016 via internal conductor 1015 and electrical plug 1040. Each chamber 1006 of the module 1004 can have a wall 1170 that can rest flush with the surface of respective receptacles 1008 of the socket 1002.

FIG. 12 is a cutaway side view of a receptacle 1208 of a socket 1202 with subsurface electrodes 1210 according to certain aspects of the present disclosure. The socket 1202 can be similar to socket 102 of FIG. 1. The socket 1202 can include electrodes 1210 positioned within the socket 1202 at a location adjacent the surface of the receptacle 1208. Electrodes 1210 can be located below the surface of the receptacle 1208. Each electrode 1210 can be coupled to internal conductors 1214.

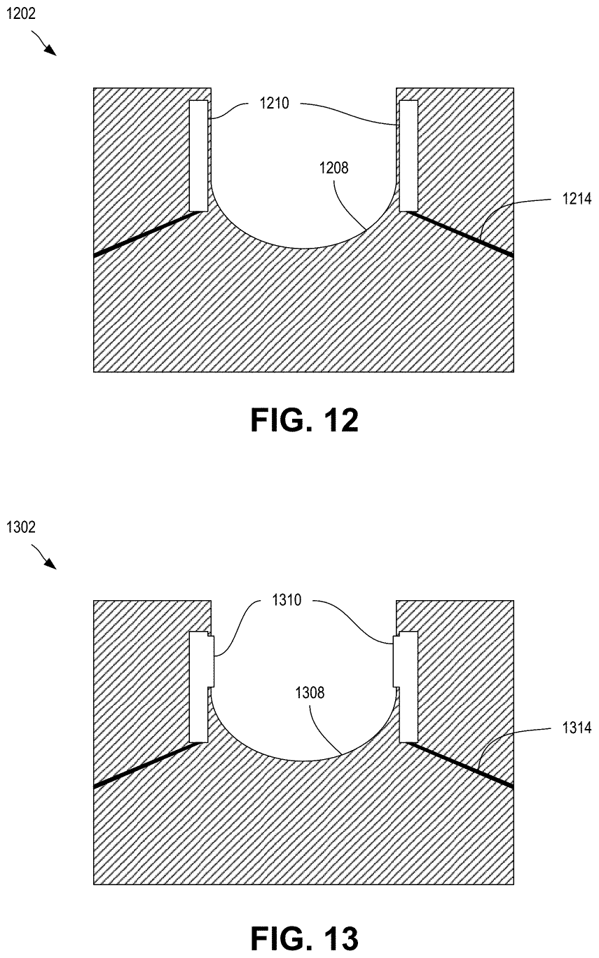

FIG. 13 is a cutaway side view of a receptacle 1308 of a socket 1302 with trans-surface electrodes 1310 according to certain aspects of the present disclosure. The socket 1302 can be similar to socket 102 of FIG. 1. The socket 1302 can include electrodes 1310 positioned within the socket 1302 at a location adjacent the surface of the receptacle 1308. Electrodes 1310 can extend through the surface of the receptacle 1308 such that the electrodes 1310 can make physical contact with a chamber 1306 placed within the receptacle 1308. Each electrode 1310 can be coupled to internal conductors 1314.

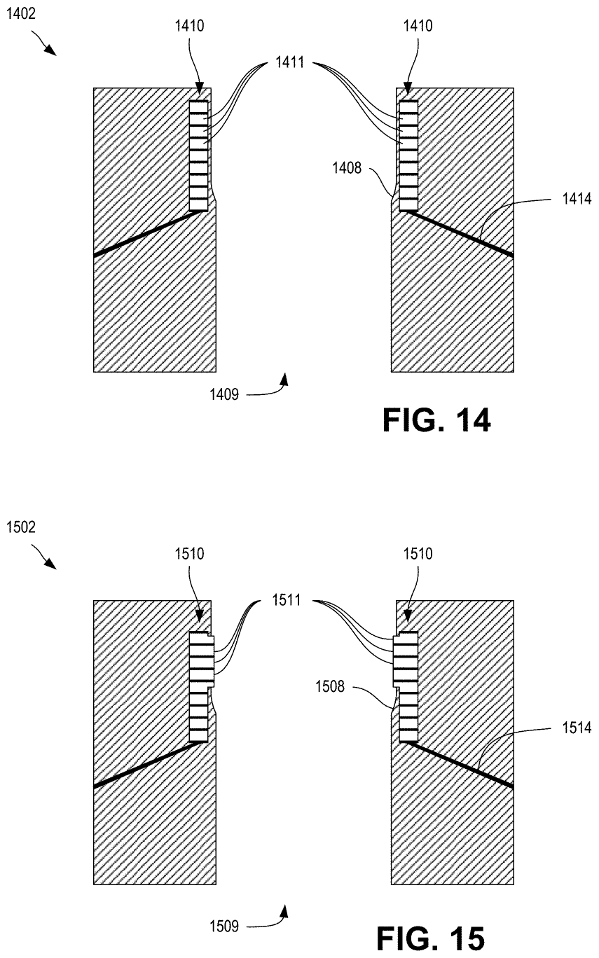

FIG. 14 is a cutaway side view of a receptacle 1408 of a socket 1402 with subsurface electrode arrays 1410 and a view port 1409 according to certain aspects of the present disclosure. The socket 1402 can be similar to socket 102 of FIG. 1. The socket 1402 can include electrode arrays 1410 positioned within the socket 1402 at a location adjacent the surface of the receptacle 1408. Electrode arrays 1410 can be located below the surface of the receptacle 1408. Each electrode array 1410 can be coupled to internal conductors 1414.

Each electrode array 1410 can include one or more electrodes 1411. As seen in FIG. 14, the electrode arrays 1410 each include nine electrodes 1411, although any suitable number can be used, such as 1, 2, 3, 4, 5, 6, 7, 8, 9, 10, or more. In some cases, the number of electrodes 1411 in an electrode array 1410 can be selected to provide desired measurement abilities, such as the ability to provide measurements for an electrocardiogram (EKG), an electroencephalogram (EEG), or an electromyogram (EMG). Such measurements can be especially useful for monitoring minibrains, cardiac organoids, and other organoids. The use of a multi-electrode electrode array 1410 can enable electrical measurements to be taken across desired paths through the receptacle 1408, and thus across desired paths of a well or chamber of a module that has been placed within receptacle 1408.

In some cases, the socket 1402 can be made of a transparent or translucent material, or can at least include sufficient transparent or translucent material adjacent a receptacle 1408 to allow for visual inspection of the contents of a well or chamber of a module that has been placed within receptacle 1408. In some cases, the socket 1402 can include view ports 1409 adjacent some or each of the receptacles 1408. The view ports 1409 can provide unobscured access to the well or chamber of a module that has been placed within receptacle 1408, allowing for visual inspection, such as using the naked eye, using a microscope, or using a camera or image sensor. View ports 1409 can be used irrespective of whether or not electrode arrays 1410 are also used. For example, a suitable view port can be used in socket 102 of FIG. 1 or elsewhere.

FIG. 15 is a cutaway side view of a receptacle 1508 of a socket 1502 with trans-surface electrode arrays 1510 and a view port 1509 according to certain aspects of the present disclosure. The socket 1502 can be similar to socket 102 of FIG. 1. The socket 1502 can include electrode arrays 1510 positioned within the socket 1502 at a location adjacent the surface of the receptacle 1508. Electrode arrays 1510 can extend through the surface of the receptacle 1508 such that the electrodes 1511 of the electrode arrays 1510 can make physical contact with a chamber 1506 placed within the receptacle 1508. Each electrode array 1510 can be coupled to internal conductors 1514.

Each electrode array 1510 can include one or more electrodes 1511. As seen in FIG. 15, the electrode arrays 1510 each include nine electrodes 1511, although any suitable number can be used, such as 1, 2, 3, 4, 5, 6, 7, 8, 9, 10, or more. In some cases, the number of electrodes 1511 in an electrode array 1510 can be selected to provide desired measurement abilities, such as the ability to provide electrocardiogram (EKG) measurements or electroencephalogram (EEG) measurements. Such measurements can be especially useful for monitoring minibrains, cardiac organoids, and other organoids. The use of a multi-electrode electrode array 1510 can enable electrical measurements to be taken across desired paths through the receptacle 1508, and thus across desired paths of a well or chamber of a module that has been placed within receptacle 1508.

In some cases, the socket 1502 can be made of a transparent or translucent material, or can at least include sufficient transparent or translucent material adjacent a receptacle 1508 to allow for visual inspection of the contents of a well or chamber of a module that has been placed within receptacle 1508. In some cases, the socket 1502 can include view ports 1509 adjacent some or each of the receptacles 1508. The view ports 1509 can provide unobscured access to the well or chamber of a module that has been placed within receptacle 1508, allowing for visual inspection, such as using the naked eye, using a microscope, or using a camera or image sensor. View ports 1509 can be used irrespective of whether or not electrode arrays 1510 are also used. For example, a suitable view port can be used in socket 102 of FIG. 1 or elsewhere.

The foregoing description of the embodiments, including illustrated embodiments, has been presented only for the purpose of illustration and description and is not intended to be exhaustive or limiting to the precise forms disclosed. Numerous modifications, adaptations, and uses thereof will be apparent to those skilled in the art.

As used below, any reference to a series of examples is to be understood as a reference to each of those examples disjunctively (e.g., “Examples 1-4” is to be understood as “Examples 1, 2, 3, or 4”).

Example 1 is a microfluidic system comprising a microfluidic module having an array of chambers fluidly coupled to a set of fluid interface elements of the microfluidic module; a socket for receiving the microfluidic module, the socket including: a plurality of fluid ports in fluid communication with a set of fluid interface elements of the socket, wherein the set of fluid interface elements of the socket are fluidly couplable to the set of fluid interface elements of the microfluidic module; an array of receptacles shaped to accept respective chambers of the array of chambers of the microfluidic module; a plurality of electrical ports electrically coupled to a plurality of electrodes positioned at respective receptacles of the array of receptacles.

Example 2 is the system of example 1, wherein the set of fluid interface elements of the microfluidic module include a set of recessed passageways, and wherein the set of fluid interface elements of the socket include a set of rigid passageways insertable into the set of recessed passageways to fluidly couple the plurality of fluid ports of the socket to the microfluidic module.

Example 3 is the system of example 2, wherein each of the set of rigid passageways includes a protuberance capable of engaging respective recessed passageways of the set of recessed passageways to facilitate a fluid-tight seal.

Example 4 is the system of examples 1-3, wherein each of the plurality of electrodes extends through respective surfaces of respective receptacles of the array of receptacles.

Example 5 is the system of examples 1-4, wherein the plurality of electrodes are coupled to the plurality of electrical ports by electrical conductors, and wherein the plurality of electrodes and the electrical conductors are positioned in the socket at locations between receptacles of the array of receptacles.

Example 6 is the system of examples 1-5, wherein the plurality of electrodes are made of a transparent material.

Example 7 is the system of examples 1-6, wherein the socket further includes an electrical interface element couplable to an electrical interface element of the microfluidic module, and wherein the electrical interface element of the socket is in electrical communication with at least one of the plurality of electrical ports.

Example 8 is the system of examples 1-7, wherein the microfluidic module and the socket are made of transparent materials.

Example 9 is the system of examples 1-8, further comprising a second microfluidic module interchangeable with the microfluidic module.

Example 10 is a method, comprising coupling a first microfluidic module to a socket, wherein the first microfluidic module includes a chamber; flowing fluid through the socket and the chamber of the first microfluidic module using a fluid interface between the socket and the first microfluidic module; sensing or applying electrical current through the chamber of the first microfluidic module using electrodes positioned in the socket; and decoupling the first microfluidic module from the socket.

Example 11 is the method of example 10, further comprising coupling a second microfluidic module to the socket, wherein the second microfluidic module includes a chamber; flowing fluid through the socket and the chamber of the second microfluidic module using a fluid interface between the socket and the second microfluidic module; and sensing or applying electrical current through the chamber of the second microfluidic module using electrodes positioned in the socket.

Example 12 is the method of examples 10 or 11, wherein the fluid interface includes a rigid passageway of the socket insertable into a recessed passageway of the first microfluidic module.

Example 13 is the method of example 12, wherein coupling the first microfluidic module to the socket includes creating a fluid-tight seal using a protuberance of the rigid passageway.

Example 14 is the method of examples 10-13, wherein sensing or applying electrical current through the chamber of the first microfluidic module includes sensing electrical activity of cells within the chamber.

Example 15 is the method of examples 10-14, wherein sensing or applying electrical current through the chamber of the first microfluidic module includes applying an electrical stimulus to cells within the chamber.

Example 16 is the method of examples 10-15, further comprising transmitting light through the socket and the chamber.

Example 17 is a microfluidic socket, comprising a set of fluid ports in fluid communication with a set of fluid interface elements, each of the set of fluid interface elements being couplable to channels of a module to fluidly couple the set of fluid ports to a chamber of the module; a receptacle shaped to accept the chamber of the module; a set of electrical ports in electrical communication with electrodes positioned adjacent the receptacle to conduct electricity through the chamber of the module and the set of electrical ports.

Example 18 is the socket of example 17, wherein the set of fluid interface elements includes a set of rigid passageways insertable into respective recessed passageways of the module.

Example 19 is the socket of examples 17 or 18, wherein the electrodes extend through a surface of the receptacle.

Example 20 is the socket of examples 17-19, wherein the electrodes are made of transparent materials.

What is claimed is:

1. A microfluidic system comprising:
a microfluidic module having an array of chambers fluidly coupled to a set of fluid interface elements of the microfluidic module;
a socket for receiving the microfluidic module, the socket including:
a plurality of fluid ports in fluid communication with a set of fluid interface elements of the socket, wherein the set of fluid interface elements of the socket are fluidly couplable to the set of fluid interface elements of the microfluidic module;
an array of receptacles shaped to accept respective chambers of the array of chambers of the microfluidic module;
a plurality of electrical ports electrically coupled to a plurality of electrodes positioned at respective receptacles of the array of receptacles.

2. The system of claim 1, wherein the set of fluid interface elements of the microfluidic module include a set of recessed passageways, and wherein the set of fluid interface elements of the socket include a set of rigid passageways insertable into the set of recessed passageways to fluidly couple the plurality of fluid ports of the socket to the microfluidic module.

3. The system of claim 2, wherein each of the set of rigid passageways includes a protuberance capable of engaging respective recessed passageways of the set of recessed passageways to facilitate a fluid-tight seal.

4. The system of claim 1, wherein each of the plurality of electrodes extends through respective surfaces of respective receptacles of the array of receptacles.

5. The system of claim 1, wherein the plurality of electrodes are coupled to the plurality of electrical ports by electrical conductors, and wherein the plurality of electrodes and the electrical conductors are positioned in the socket at locations between receptacles of the array of receptacles.

6. The system of claim 1, wherein the plurality of electrodes are made of a transparent material.

7. The system of claim 1, wherein the socket further includes an electrical interface element couplable to an electrical interface element of the microfluidic module, and wherein the electrical interface element of the socket is in electrical communication with at least one of the plurality of electrical ports.

8. The system of claim 1, wherein the microfluidic module and the socket are made of transparent materials.

9. The system of claim 1, further comprising a second microfluidic module interchangeable with the microfluidic module.

10. A method, comprising:
coupling a first microfluidic module to a socket, wherein the first microfluidic module includes a chamber;
flowing fluid through the socket and the chamber of the first microfluidic module using a fluid interface between the socket and the first microfluidic module;
sensing or applying electrical current through the chamber of the first microfluidic module using electrodes positioned in the socket; and
decoupling the first microfluidic module from the socket.

11. The method of claim 10, further comprising:
coupling a second microfluidic module to the socket, wherein the second microfluidic module includes a chamber;
flowing fluid through the socket and the chamber of the second microfluidic module using a fluid interface between the socket and the second microfluidic module; and
sensing or applying electrical current through the chamber of the second microfluidic module using electrodes positioned in the socket.

12. The method of claim 10, wherein the fluid interface includes a rigid passageway of the socket insertable into a recessed passageway of the first microfluidic module.

13. The method of claim 12, wherein coupling the first microfluidic module to the socket includes creating a fluid-tight seal using a protuberance of the rigid passageway.

14. The method of claim 10, wherein sensing or applying electrical current through the chamber of the first microfluidic module includes sensing electrical activity of cells within the chamber.

15. The method of claim 10, wherein sensing or applying electrical current through the chamber of the first microfluidic module includes applying an electrical stimulus to cells within the chamber.

16. The method of claim 10, further comprising transmitting light through the socket and the chamber.

17. A microfluidic socket, comprising:
a set of fluid ports in fluid communication with a set of fluid interface elements, each of the set of fluid interface elements being couplable to channels of a module to fluidly couple the set of fluid ports to a chamber of the module;
a receptacle shaped to accept the chamber of the module;
a set of electrical ports in electrical communication with electrodes positioned adjacent the receptacle to conduct electricity through the chamber of the module and the set of electrical ports.

18. The socket of claim 17, wherein the set of fluid interface elements includes a set of rigid passageways insertable into respective recessed passageways of the module.

19. The socket of claim 17, wherein the electrodes extend through a surface of the receptacle.

20. The socket of claim 17, wherein the electrodes are made of transparent materials.
